(19) United States
(12) Patent Application Publication
(10) Pub. No.: US 2015/0353486 A1
(43) Pub. Date: Dec. 10, 2015

(54) Difluorocarbene From Fluoroform for Preparation of Difluoromethyoxyarenes, Difluorothiomethoxyarenes and Heteroarenes

(71) Applicant: UNIVERSITY OF FLORIDA RESEARCH FOUNDATION, INC., Gainesville,, FL (US)

(72) Inventors: CHARLES S. THOMOSON, TIFTON, GA (US); WILLIAM R. DOLBIER, JR., GAINESVILLE, FL (US)

(21) Appl. No.: 14/759,026

(22) PCT Filed: Jan. 15, 2014
(86) PCT No.: PCT/US14/11570

§ 371 Date: Jul. 2, 2015

Related U.S. Application Data
(60) Provisional application No. 61/752,578, filed on Jan. 15, 2013.

(51) Int. Cl. C07C 319/14; C07C 41/01

(52) U.S. Cl. CPC C07C 319/14; C07C 41/01

(57) ABSTRACT

A method for the transformation of hydroxyarenes, hydroxyheteroarenes, thiohydroxyarenes or thiohydroxyheteroarenes involves reaction with fluoroform in the presence of base comprising solution to yield a difluoromethoxyarene, diflurothiomethoxyheteroarene, or diflurothiomethoxyheteroarene. The transformation can be carried out at ambient room pressure or at elevated pressures. The transformation can be carried out at temperatures of 0 to 70° C.

### CROSS-REFERENCE TO RELATED APPLICATION

The present application claims the benefit of U.S. Provisional Application Ser. No. 61/752,578, filed Jan. 15, 2013, which is hereby incorporated by reference herein in its entirety, including any figures, tables, or drawings.

### BACKGROUND OF INVENTION

Fluoroform or trifluoromethane (CF₃H) is a side-product in the manufacture of Teflon. It is available in large quantities and is nontoxic, non-ozone-depleting and inexpensive. Fluoroform is used in the semiconductor industry for plasma etching of silicon oxide and silicon nitride. It is useful as a refrigerant (R-23 or HFC-23) for replacement for chlorotrifluoromethane (cfc-13). It can be used as a fire suppressant, and has been used as a replacement for Halon 1301 (cfc-13b1) in fire suppression systems as a total flooding gaseous agent.

As a reagent, CHF₃ is used to generate the anion CF3⁻ by deprotonation and as a precursor to CF₃Si(CH₃)₃. Fluoroform is potentially an ideal source to prepare stable trifluromethylmetal derivatives. Although, fluoroform can be deprotonated by strong bases, such as KCH₂S(O)Me, the resulting anion is notorious for facile decomposition to difluorocarbene at room temperature or below. This decomposition is avoided by deprotonating CF₃H at −20 to −40° C. in DMF such that addition across the carbonyl yields Me₂NCH(OK)CF₃.

Difluoromethyl ethers are found as pharmaceuticals, agrochemicals, and materials. Aryl difluoromethyl ethers are medicinally important as enzyme inhibitors, anti-HIV agents and antimicrobial agents. For example, pantoprazole is a proton-pump inhibitor that is a difluoromethyloxyarene. Current syntheses of difluoromethyl ethers require the use of HCF₂Cl, which is difficult to handle because it is an ozone depleting gas and elevated temperatures are required for the transformation. Other sources of the HCF₂ group react only at high temperatures and typically require long reaction periods. More recently, Fier et al. Angew. Chem. Int. Ed. 2013, 52, 1, has reported the use of difluoromethyltriflate, an air stable liquid, as the source of a difluoromethyl groups. Difluoromethyltriflate can be prepared from CF₃Si(CH₃)₃ and trifluoromethanesulfonic acid (triflic acid) at room temperature in the presence of TiCl₄ in good yields This reagent has shown to be useful for the preparation of difluoromethoxyarenes and hetereoarenes rapidly at room temperature in a mixed solvent in good yield, although co-formation of an aryltriflate side-product occurs. The use of the triflate increases the cost of synthesis. The formation of the triflate side-product complicates the isolation of the desired product.

Hence, the production of difluoromethoxyarenes and hetereoarenes at a reasonably rapid rate under low to moderate pressures and temperatures with an inexpensive difluoromethene equivalent remains desirable.

### BRIEF SUMMARY

A method of using fluoroform as a source of difluorocarbene for the preparation of difluromethoxyarenes, difluorothiomethoxyarenes, difluoromethoxyhetereoarenes, and difluorothiomethoxyheteroarenes involves low and moderate temperatures and pressures. The method involves the reaction of a phenol, substituted phenol, thiophenol, substituted thiophenol, or other hydroxy or thiohydroxy substituted arene, such as a hydroxy substituted heteroarene, thiohydroxy substituted heteroarene , hydroxy substituted polycyclic arene, or thiohydroxy substituted polycyclic arene. The reaction can occur in a mixed solvent including water. A base is employs for the generation of difluorocarbene.

### BRIEF DESCRIPTION OF DRAWINGS

FIG. 1 is a proton decoupled ¹⁹F NMR spectrum of difluoromethoxybenzene prepared from fluoroform, according to an embodiment of the invention.

FIG. 2 is a proton coupled ¹⁹F NMR spectrum of difluoromethoxybenzene prepared from fluoroform, according to an embodiment of the invention.

### DETAILED DISCLOSURE

An embodiments of the invention is directed to a method of the reaction of fluoroform with phenols and thiophenols to form difluoromethoxybenzenes and difluorothiomethoxybenzenes. Other embodiments of the invention are directed to the preparation of difluoromethoxyheteroarenes and difluorothiomethoxyheteroarenes. The exemplary transformation of phenol and thiophenol to difluoromethoxybenzene and difluorothiomethoxybenzene are shown below in Equations (1) and (2):

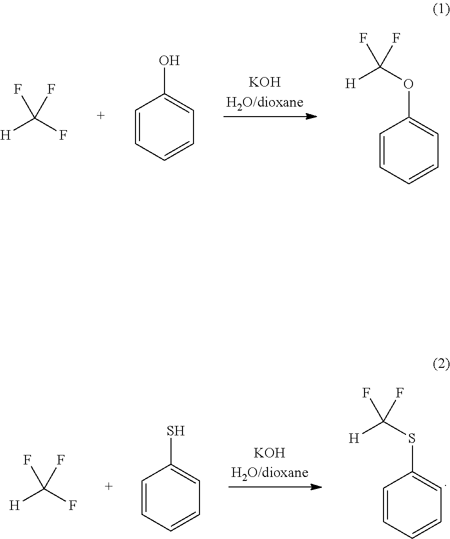

The method can be carried out in good yield at atmospheric pressure in a two-phase mixture, such as water/dioxane, with a base, such as KOH, in a relatively short period of time. For example, the desired transformation can occur in a few hours, by simply contacting a fluoroform atmosphere with the solution of the phenol or thiophenol at temperatures of about 0 to about 70° C., or higher. In other embodiments of the invention, a one-phase solution where the phenol or thiophenol and base are soluble can be used. The method can be carried out at atmospheric pressure, and can use a circulation or recirculation of fluoroform vapor through the two-phase mixture or one-phase solution, or can he carried out at a pressure inherent to that of fluoroform in a closed system at a desired reaction temperature, for example, 4.4 MPa at 20° C.

For purposes of the invention, the “phenol” or “thiophenol” can refer to any hydroxy or thiohydroxy substituted: monocyclic aromatic; monocyclic heteroaromatic; polycyclic aromatic; or polycyclic heteroaromatic compound. For purposes of the invention, the “phenol” or “thiophenol” can be substituted or plurally substituted with alkyl, aryl, alkenyl, alkynyl, halo, alkoxy, aryloxy, amino, amido, imido, cyano, nitro, or any other substituent that can tolerate basic conditions at room temperatures without significant side reaction over the time required for reaction of fluoroform with the OH or SH functionality. The “phenol” or “thiophenol” can be a plurally OH and/or SH substituted compound, where reaction results in the transformation of one or more OH or SH difluormethoxy or difluorothiomethoxy substituents. The product of the synthetic method, according to an embodiment of the invention, can be any mono-, di- or poly- difluoromethoxyarene and/or difluorothiomethoxyarene or heteroarenes. The “phenol” or “thiophenol” can be a natural product or a compound derived from a natural product.

In an embodiment of the invention, the use of a two-phase solvent mixture allows the separation of the product difluoromethoxy or difluorothiomethoxy arene in an organic phase and unreacted phenol or thiophenol in its deprotonated form. The desired product can be isolated by any method including: distillation; sublimation; crystallization; or chromatography. In an embodiment of the invention, the base can be a basic ion exchange resin or other base that can be separated by filtration from the product solution upon completion of the reaction.

### Methods and Materials

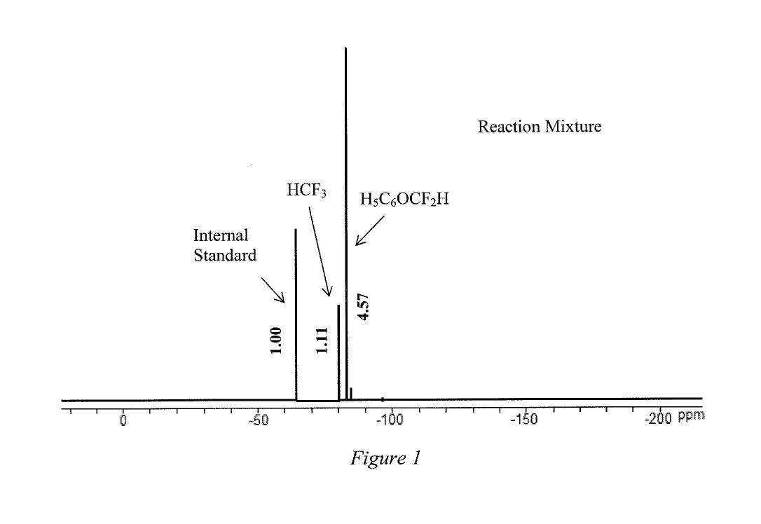

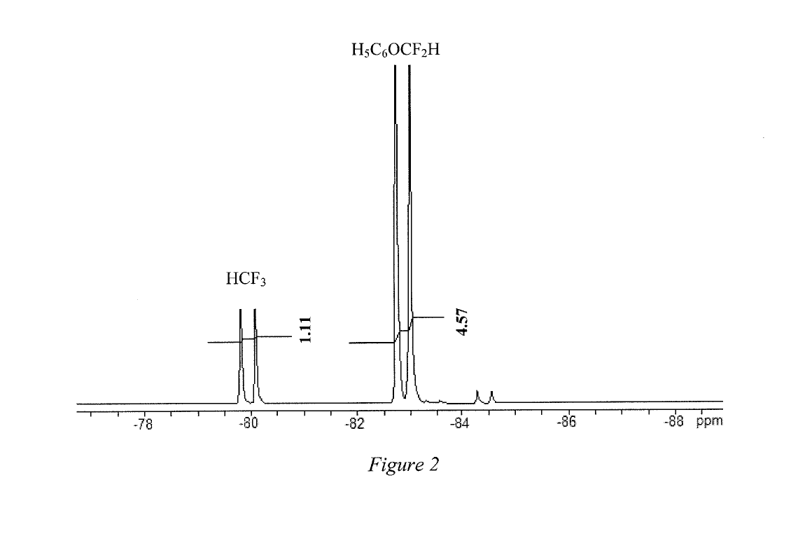

In a 50 mL round-bottomed flask was placed 0.28 g (0.003 moles) of phenol, 1.7 g of KOH (0.03 moles), with water and dioxane at room temperature. The mixture was heated to 50° C., fluoroform was bubbled through the mixture for two hours, and the mixture maintained at 50° C. overnight. FIGS. 1 and 2 show the ¹⁹F NMR decoupled and proton-coupled spectra of the reaction mixture and separated difluoromethoxybenzene, respectively. A difluoromethoxybenzene yield of 70% was determined from the ¹⁹F NMR integrated signal for the HF₂CO group relative to an internal standard.

In a 50 mL round-bottomed flask was placed 0.31 mL (0.003 moles) of thiophenol, 1.7 g of KOH (0.03 moles) with water and dioxane at room temperature. The mixture was heated to 50° C., fluoroform was bubbled through the mixture for two hours, and the mixture maintained at 50° C. overnight. A difluorothiomethoxybenzene yield of 55% was determined from the ¹⁹F NMR integrated signal for the HF₂CS group relative to an internal standard.

In a 50 mL round-bottomed flask was placed 0.28 g (0.003 moles) of phenol with 3.4 g of KOH (0.060 moles) in water and dioxane at room temperature. The mixture was heated to 50° C., fluoroform was bubbled through the mixture for two hours, and the mixture maintained at 50° C. overnight. A difluoromethoxybenzene yield of 48% was determined from the ¹⁹F NMR integrated signal for the HF₂CO group relative to an internal standard.

In a 50 mL round-bottomed flask was placed 0.31 mL (0.003 moles) of thiophenol with 3.4 g of KOH (0.03 moles) in water and dioxane at room temperature. The mixture was heated to 50° C., fluoroform was bubbled through the mixture for two hours, and the mixture maintained at 50° C. overnight. A difluorothiomethoxybenzene yield of 65% was determined from the ¹⁹F NMR integrated signal for the HF₂CS group relative to an internal standard.

All publications referred to or cited herein are incorporated by reference in their entirety, including all figures and tables, to the extent they are not inconsistent with the explicit teachings of this specification.

It should be understood that the examples and embodiments described herein are for illustrative purposes only and that various modifications or changes in light thereof will be suggested to persons skilled in the art and are to be included within the spirit and purview of this application.

We claim:

1. A method of preparing a difluoromethoxyarene or difluorothiomethoxyarene comprising:
providing a hydroxyarene or thiohydroxyarene;
combining the hydroxyarene or thiohydroxyarene with a base and a solvent to form a reaction solution; and
adding fluoroform to the reaction solution, wherein at least a portion of the hydroxyarene or thiohydroxyarene is converted into a difluoromethoxyarene or a difluorothiomethoxyarene.

2. The method of claim 1, wherein the hydroxyarene is a hydroxyheteroarene.

3. The method of claim 1, wherein the thiohydroxyarene is a thiohydroxyheteroarene.

4. The method of claim 1, wherein the temperature of the reaction solution is 0 to 70° C.

5. The method of claim 1, wherein the pressure on the reaction solution is 1 atmosphere.

6. The method of claim 1, wherein the solvent is a mixed solvent comprising water.

7. The method of claim 6, wherein the mixed solvent further comprises dioxane or other ethereal solvent.

8. The method of claim 1, further comprising isolating the difluoromethoxyarene or the difluorothiomethoxyarene.

9. The method of claim 8, wherein isolating comprises distilling, subliming, crystallizing, or chromatographically separating.
